(19) United States
(12) Patent Application Publication
(10) Pub. No.: US 2026/0284403 A1
(43) Pub. Date: Sep. 24, 2026

(54) STIMULATION CONTROL

(71) Applicant: Cochlear Limited, Macquarie University, NSW (AU)

(72) Inventors: Timothy Jean BROCHIER, East Melbourne (AU); Zachary Mark SMITH, Pymble (AU); Adam HERSBACH, Richmond (AU); Brett Anthony SWANSON, St. Ives (AU)

(21) Appl. No.: 19/473,382

(22) PCT Filed: Apr. 5, 2024
(86) PCT No.: PCT/IB2024/053361

§ 371 Date: Oct. 7, 2025

Related U.S. Application Data
(60) Provisional application No. 63/495,896, filed on Apr. 13, 2023.

(51) Int. Cl. A61N 1/36

(52) U.S. Cl. CPC A61N 1/36139; A61N 1/36038

(57) ABSTRACT

Presented herein are techniques for controlling stimulation provided by an implantable device. The stimulation control can be performed at an external device, and can machine learning (e.g., artificial intelligence (AI)). The techniques provide a cochlear implant stimulation strategy that utilizes computational models of a healthy auditory system and an implanted auditory system to more closely emulate natural acoustic hearing.

### BACKGROUND

### Field of the Invention

Aspects of the present invention relate generally to controlling stimulation delivered by an electronic device.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, Fully-implantable vision prostheses, vagal nerve stimulators, spinal cord stimulators, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: receiving signals associated with a physiological function at an implantable medical device system; determining, by a machine learning model based on the signals, information for stimulation signals for stimulation of the physiological function, wherein the machine learning model is trained based on modelling physiological effects from the stimulation; and controlling the stimulation to a recipient of the implantable medical device system based on the determined information.

In another aspect, one or more non-transitory computer readable storage media comprising instructions are provided. The instructions, when executed by one or more processors, cause the one or more processors to: receive signals associated with a physiological function at an implantable medical device system; determine, by a machine learning model based on the signals, information for stimulation signals for stimulation of the physiological function, wherein the machine learning model is trained based on modelling physiological effects from the stimulation; and control the stimulation to a recipient of the implantable medical device system based on the determined information.

In another aspect, an implantable medical device system is provided. The implantable medical device system comprises: memory for storing data; and one or more processors, wherein the one or more processors are configured to: receive signals associated with a physiological function; determine, by a machine learning model based on the signals, information for stimulation signals for stimulation of the physiological function, wherein the machine learning model is trained based on modelling physiological effects from the stimulation; and control the stimulation to a recipient of the implantable medical device system based on the determined information.

In another aspect, another method is provided. The method comprises: determining, by a machine learning model of at least one processor based on signals associated with a physiological function, information of stimulation signals for stimulation of the physiological function; modelling, via the at least one processor, physiological effects from the stimulation signals; and updating, via the at least one processor, the machine learning model based on a difference between the modelled physiological effects and reference physiological effects representing normal physiological function.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2 is a functional block diagram illustrating an example audio signal processing path of a cochlear implant system with which aspects of the techniques presented herein can be implemented;

FIG. 3 is a functional block diagram illustrating a method of training a hearing machine learning (ML) model according to certain embodiments;

FIG. 4A illustrates an example spectrogram for a rising complex tone;

FIG. 4B illustrates an example neurogram of inner hair cell voltage for the rising complex tone of FIG. 4A;

FIG. 4C illustrates an example neurogram of fine structure of an auditory nerve for the rising complex tone of FIG. 4A;

FIG. 5 is a functional block diagram illustrating a method of training a sound processor machine learning (ML) model for controlling stimulation according to certain embodiments;

FIG. 6 is a schematic diagram of an example neural network with which aspects of the techniques presented herein can be implemented;

FIG. 7 is a functional block diagram illustrating a method of training a sound processor machine learning (ML) model using feature extraction applied to an input audio signal according to certain embodiments;

FIG. 8 is a functional block diagram illustrating a method of training a sound processor machine learning (ML) model with another feature extraction applied to an input audio signal according to certain embodiments;

FIG. 9 is a functional block diagram illustrating a method of training a sound processor machine learning (ML) model with a neurogram of an input audio signal according to certain embodiments;

FIG. 10 is a functional block diagram illustrating a method of training a sound processor machine learning (ML) model to reduce noise according to certain embodiments;

FIG. 11A is a functional block diagram illustrating a method of training a sound processor machine learning (ML) model for focused multi-polar stimulation with a neurogram of an input audio signal according to certain embodiments;

FIG. 11B is a functional block diagram illustrating another method of training a sound processor machine learning (ML) model for focused multi-polar stimulation with a neurogram of an input audio signal according to certain embodiments;

FIG. 12 is a flowchart illustrating an example process to control stimulation according to certain embodiments; and

FIG. 13 is a flowchart illustrating an example process for training a machine learning model to control stimulation according to certain embodiments.

### DETAILED DESCRIPTION

Presented herein are techniques for controlling stimulation provided by an electronic device, such as an implantable medical device. The stimulation control can be performed at an external device, and can use machine learning (e.g., artificial intelligence (AI)). In certain aspects, the techniques provide a stimulation strategy that utilizes computational models of a healthy auditory system and an implanted auditory system, to more closely emulate natural acoustic hearing.

Exemplary techniques presented herein minimize a difference between normal hearing and electrical hearing computational models to transmit sound information that is more consistent with normal hearing and improve outcomes for hearing device users.

It is to be appreciated that there are a number of different types of medical devices in/with which the techniques presented herein can be implemented. Merely for ease of description, the techniques presented herein are primarily described with reference to a specific medical device in the form of a cochlear implant. However, it is to be appreciated that the techniques presented herein can also be partially or fully implemented by any of a number of different types of medical devices, particularly other types of devices for delivery of electrical stimulation signals to a recipient. For example, the techniques can be implemented in devices delivering electrical stimulation to the auditory nerve, middle ear, vestibular system, retina and/or brain, among other regions of the body. As used herein, the term “hearing device” is to be broadly construed as any device that delivers sound signals to a user in any form, including in the form of acoustical stimulation, mechanical stimulation, electrical stimulation, optical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems. combinations or variations thereof, etc.) or a device for use by a person with normal hearing (e.g., consumer devices that provide audio streaming, consumer headphones, earphones and other listening devices).

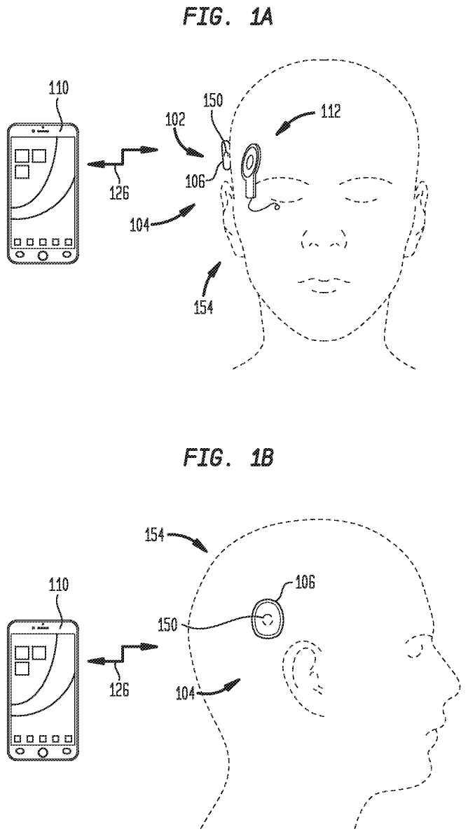

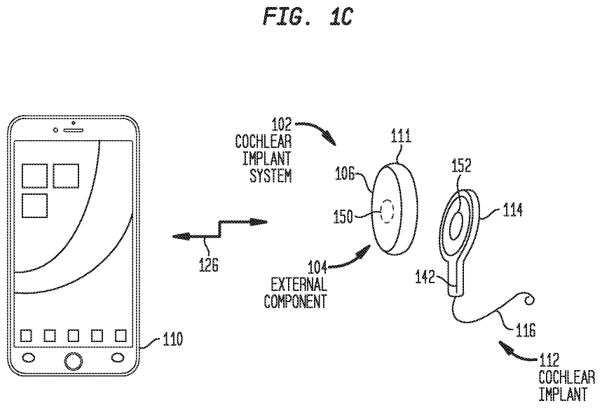

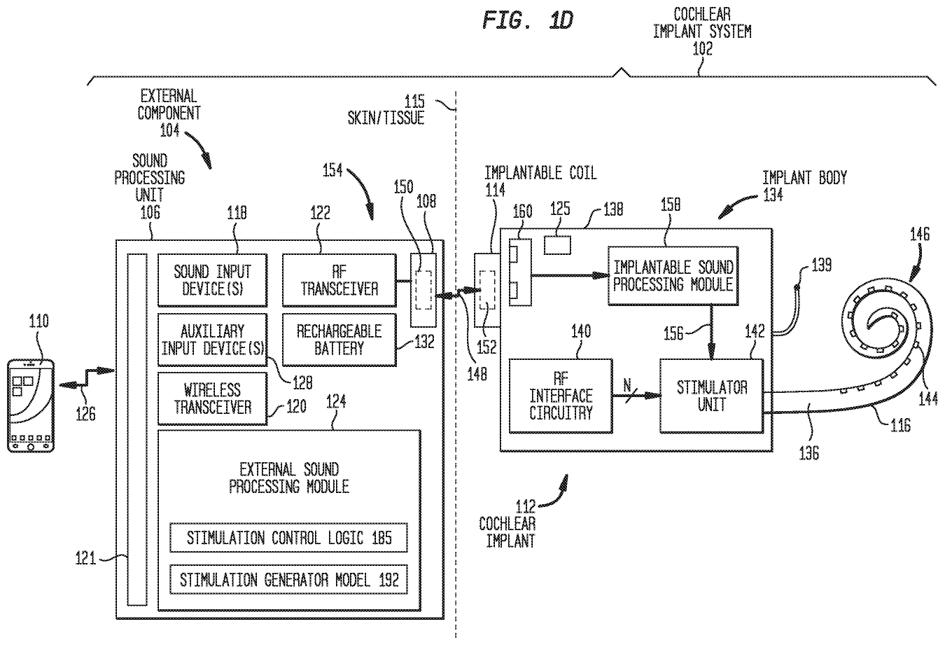

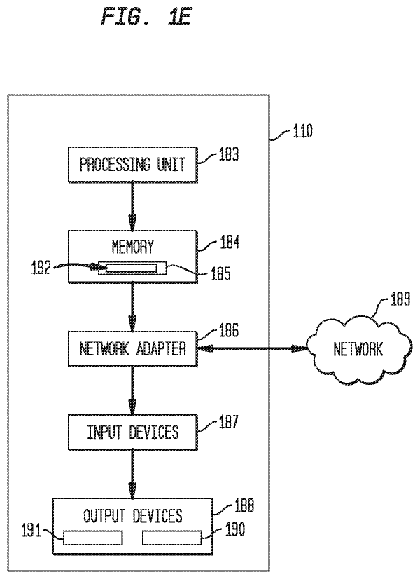

FIGS. 1A-1E illustrate an example cochlear implant system 102 with which aspects of the techniques presented herein can be implemented. The cochlear implant system 102 comprises an external component 104 and an implantable component 112. In the examples of FIGS. 1A-1E, the implantable component is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1E will generally be described together.

Cochlear implant system 102 includes an external component 104 that is configured to be directly or indirectly attached to the body of the user and an implantable component (or implant) 112 configured to be implanted in the user. In the examples of FIGS. 1A-1E, the external component 104 comprises a sound processing unit 106, while the cochlear implant 112 includes an implantable coil 114, an implant body 134, and an elongate stimulating assembly 116 configured to be implanted in the user's cochlea.

In the example of FIGS. 1A-1E, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head (e.g., includes an integrated external magnet 150 configured to be magnetically coupled to an implantable magnet 152 in the implantable component 112). The OTE sound processing unit 106 also includes an integrated (headpiece) coil 108 that is configured to be inductively coupled to the implantable coil 114.

It is to be appreciated that the OTE sound processing unit 106 is merely illustrative of the external devices that can operate with implantable component 112. For example, in alternative examples, the external component can comprise a behind-the-ear (BTE) sound processing unit or a micro-BTE sound processing unit and a separate external coil assembly. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components can be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. In certain examples, in the invisible hearing mode, an external device can still deliver power to the implant. In such examples, the external device can implement the techniques presented herein to use information (e.g., stimulation parameters) from the cochlear implant 112, retrieved or stored on the external device, to calculate an optimum power level. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 can also operate in alternative modes.

In FIGS. 1A and 1C, the cochlear implant system 102 is shown with an external computing device 110, configured to implement aspects of the techniques presented. The computing device 110, which is shown in greater detail in FIG. 1E, is, for example, a personal computer, server computer, hand-held device, laptop device, multiprocessor system, microprocessor-based system, programmable consumer electronic (e.g., smart phone), network PC, minicomputer, mainframe computer, tablet, remote control unit, distributed computing environment that include any of the above systems or devices, and the like. The computing device 110 can be a single virtual or physical device operating in a networked environment over communication links to one or more remote devices, such as an implantable medical device or implantable medical device system.

In its most basic configuration, computing device 110 includes at least one processing unit 183 and memory 184. The processing unit 183 includes one or more hardware or software processors (e.g., Central Processing Units) that can obtain and execute instructions. The processing unit 183 can communicate with and control the performance of other components of the computing device 110.

The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include RAM, ROM, EEPROM (Electronically-Erasable Programmable Read-Only Memory), flash memory, optical disc storage, magnetic storage, solid state storage, or any other memory media usable to store information for later access. In examples, the memory 184 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 184 can include wired media such as a wired network or direct-wired connection, and wireless media such as acoustic, RF, infrared and other wireless media or combinations thereof. In certain embodiments, the memory 184 comprises stimulation control logic 185 (with stimulation generator model 192) that, when executed, enables the processing unit 183 to perform aspects of the techniques presented.

In the illustrated example, the computing device 110 further includes a network adapter 186, one or more input devices 187, and one or more output devices 188. The computing device 110 can include other components, such as a system bus, component interfaces, a graphics system, a power source (e.g., a battery), among other components.

The network adapter 186 is a component of the computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. In certain examples, the one or more antennas can be shared with the charging coil 121 and/or external coil 108.

The one or more input devices 187 are devices over which the computing device 110 receives input from a user. The one or more input devices 187 can include physically-actuatable user-interface elements (e.g., buttons, switches, or dials), touch screens, keyboards, mice, pens, and voice input devices, among others input devices.

The one or more output devices 188 are devices by which the computing device 110 is able to provide output to a user. The output devices 188 can include, for example, a display 190 and one or more speakers 191, among other output devices.

It is to be appreciated that the arrangement for computing device or system 110 shown in FIG. 1E is merely illustrative and that aspects of the techniques presented herein can be implemented at a number of different types of systems/devices. For example, the computing device 110 can be a laptop computer, tablet computer, mobile phone, surgical system, etc.

The OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external computing device 110). However, it is to be appreciated that one or more input devices can include additional types of input devices and/or less input devices (e.g., the wireless short range radio transceiver 120 and/or one or more auxiliary input devices 128 can be omitted).

The OTE sound processing unit 106 also comprises the external coil 108, a charging coil 121, a closely-coupled transmitter/receiver (RF transceiver) 122, sometimes referred to as radio-frequency (RF) transceiver 122, at least one rechargeable battery 132, and an external sound processing module 124. The external sound processing module 124 can comprise, for example, one or more processors and a memory device (memory) that includes sound processing logic. The memory device can further include stimulation control logic 185 that, when executed, enables the one or more processors to perform aspects of the techniques presented. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic and stimulation control logic 185 (with stimulation generator model 192) stored in the memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which could potentially include at least one battery 125, RF interface circuitry 140, and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit power, and optionally data, to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user. The external sound processing module 124 can further control stimulation provided by the implant 112 preferably using machine learning (e.g., artificial intelligence (AI)) according to techniques presented herein.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode, the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in the memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 can operate differently in different embodiments. For example, in one alternative implementation of the external hearing mode, the cochlear implant 112 can use signals captured by the sound input devices 118 and the implantable sound sensors 160 in generating stimulation signals for delivery to the user.

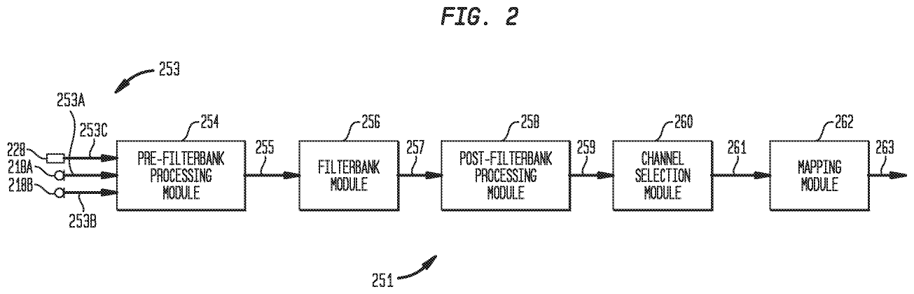

In at least one embodiment during operation of a hearing device system including a cochlear implant, as discussed in further detail below with reference to FIG. 2, sound processing module 124 is configured to convert output signals received from the input devices (e.g., one or more sound input devices 118 and/or one or more auxiliary input devices 128) into a set of output signals representative of electrical stimulation.

With reference to FIG. 2, shown is a functional block diagram illustrating an example sound/audio signal processing path of an auditory prosthesis, such as cochlear implant system 102, with which aspects of the techniques presented herein can be implemented. Various sound processing operations discussed for FIG. 2 can be performed via sound processing logic provided for any combination of an external component or an internal component of a cochlear implant system. Various features of cochlear implant system 102 as noted for FIGS. 1A-1D are discussed with reference to various features illustrated in FIG. 2.

Consider, with reference to FIG. 2, a sensory/environmental signal or audio signal processing path 251 which can be provided via sound processing module 124 of external component 104 and/or via sound processing module 158 of implantable component 112. In the example of FIG. 2, input devices can include two sound input devices, namely a first microphone 218A and a second microphone 218B, as well as at least one auxiliary input device 228 (e.g., an audio input port, a cable port, a telecoil, etc.). If not already in an electrical form, the input devices can convert received/input sound signals into electrical signals 253, referred to herein as electrical sound or sensory signals, which represent the sound/sensory signals received at the input devices. The electrical sound/sensory signals 253 can include electrical sensory signal 253A from microphone 218A, electrical sensory signal 253B from microphone 218B, and electrical sensory signal 253C from auxiliary input 228.

In FIG. 2, functional operations enabled by the audio signal processing path (i.e., the operations of one or more processor(s) when executing sound processing logic) are generally represented by modules 254, 256, 258, 260, and 262 which collectively comprise the audio signal processing path 251. Thus, the audio signal processing path 251 can include a pre-filterbank processing module 254, a filterbank module 256, a post-filterbank processing module 258, a channel selection module 260, and a mapping module 262, each of which are described in greater detail below. Stimulation generator model 192 can be used at, or replace, any portions of the signal processing path to produce stimulation signals (e.g., stimulation pulses, analog stimulation, etc.). Further, the stimulation generator model can receive the audio signals prior to, or processed at any point during, the signal processing path. The processed signals can provide various features as described below. For example, the stimulation generator model can receive audio signals after processing by filterbank module 256 as described below.

Consider an operational example in which electrical sound signals 253 generated by the input devices are provided to the pre-filterbank processing module 254. The pre-filterbank processing module 254 is configured to, as needed, combine the electrical sound signals 253 received from the input devices and prepare/enhance those signals for subsequent processing. The operations performed by the pre-filterbank processing module 254 can include, for example, microphone directionality operations, noise reduction operations, input mixing/combining operations, input selection/reduction operations, dynamic range control operations and/or other types of signal enhancement operations. The operations at the pre-filterbank processing module 254 generate a pre-filterbank output signal 255 that, as described further below, is the basis of further sound processing operations. The pre-filterbank output signal 255 represents the combination (e.g., mixed, selected, etc.) of the input signals (e.g., mixed, selected, etc.) received at the sound input devices at a given point in time.

In operation, the pre-filterbank output signal 255 generated by the pre-filterbank processing module 254 is provided to the filterbank module 256. The filterbank module 256 generates a suitable set of bandwidth limited channels, or frequency bins, that each includes a spectral component of the received sound/sensory signals. That is, the filterbank module 256 comprises a plurality of band-pass filters that separate the pre-filterbank output signal 255 into multiple components/channels, each one carrying a frequency sub-band of the original signal (i.e., frequency components of the received sound/sensory signal).

The channels created by the filterbank module 256 are sometimes referred to herein as sound processing, or band-pass filtered, channels, and the sound signal components within each of the sound processing channels are sometimes referred to herein as band-pass filtered signals or channelized signals. The band-pass filtered or channelized signals created by the filterbank module 256 are processed (e.g., modified/adjusted) as they pass through the audio signal processing path 251. As such, the band-pass filtered or channelized signals are referred to differently at different stages of the audio signal processing path 251. However, it will be appreciated that reference herein to a band-pass filtered signal or a channelized signal can refer to the spectral component of the received sound signals at any point within the audio signal processing path 251 (e.g., pre-processed, processed, selected, etc.).

At the output of the filterbank module 256, the channelized signals are initially referred to herein as pre-processed signals or filterbank channels 257. The number ‘n’ of filterbank channels 257 generated by the filterbank module 256 can depend on a number of different factors including, but not limited to, implant design, number of active electrodes, coding strategy, and/or recipient preference(s). In certain arrangements, twenty-two (22) channelized signals are created and the audio signal processing path 251 is said to include 22 channels.

The filterbank channels 257 are provided to the post-filterbank processing module 258. The post-filterbank processing module 258 is configured to perform a number of sound processing operations on the target filterbank channels 257. These sound processing operations include, for example, channelized gain adjustments (e.g., performed via Loudness Growth Function (LGF) processing) for hearing loss compensation (e.g., gain adjustments to one or more discrete frequency ranges of the sound signals, also referred to herein as filter channels), noise reduction operations, speech enhancement operations, etc., in one or more of the channels. After performing the sound processing operations, the post-filterbank processing module 258 outputs a plurality of processed channelized signals 259.

In the specific arrangement of FIG. 2, the audio signal processing path 251 includes a channel selection module 260. The channel selection module 260 is configured to perform a channel selection process to select, according to one or more selection rules, which of the ‘n’ channels should be used in hearing compensation. The signals selected at channel selection module 260 are represented in FIG. 2 by arrow 261 and are referred to herein as selected channelized signals or, more simply, selected signals.

In the embodiment of FIG. 2, the channel selection module 260 selects a subset ‘m’ of the ‘n’ processed channelized signals 259 for use in generation of electrical stimulation for delivery to a recipient (i.e., the sound processing channels are reduced from ‘n’ channels to ‘m’ channels). In one specific example, the ‘m’ largest amplitude channels (maxima) from the ‘n’ available combined channel signals are made, with ‘n’ and ‘m’ being programmable during initial fitting, and/or operation of the prosthesis. In one instance, this specific example can be associated with an Advanced Combination Encoder (ACE), generally, a stimulation coding strategy, such as Optimized Pitch and Language (OPAL). It is to be appreciated that different channel selection methods could be used, and are not limited to maxima selection. It is also to be appreciated that, in certain embodiments, the channel selection module 260 can be omitted. For example, certain arrangements can use a continuous interleaved sampling (CIS), CIS-based, or other non-channel selection sound coding strategy.

The audio signal processing path 251 for the instance illustrated in FIG. 2 can also include the mapping module 262, which can generate output signals 263. In one embodiment, the mapping module 262 can be configured to map (e.g., via stimulation generator model 192) the selected signals 261 (or the processed channelized signals 259 in embodiments that do not include channel selection) such that the output signals 263 correspond to a set of stimulation control signals (e.g., stimulation commands) that represent the attributes of the electrical stimulation signals that are to be delivered to a recipient so as to evoke perception of at least a portion of the received sound signals. This channel mapping can include, for example, threshold and comfort level mapping, dynamic range adjustments (e.g., compression), volume adjustments, etc., and can encompass selection of various sequential and/or simultaneous stimulation strategies.

In one embodiment, the set of stimulation control signals (stimulation commands) 263 that represent the electrical stimulation signals can be encoded for transcutaneous transmission (e.g., via an RF link) to an implantable component. As such, mapping module 262 can also be referred to as a channel mapping and encoding module and operates as an output block configured to convert the plurality of channelized signals into a plurality of stimulation control signals, from which the implantable component, via stimulator unit 142 can generate stimulation (current) signals for delivery to the recipient via a stimulating assembly 116.

In one embodiment, for example if channel selection module 260 is omitted from the audio signal processing path 251, the mapping module 262 can perform mapping operations that involve mapping channel envelopes to current levels, which can be mixed with streams received from one or more sources. Generally, a channel envelope is a “temporal envelope” that is extracted from each frequency band (channel) and is used to modulate pulse trains that are delivered to an implanted electrode. Thus, amplitudes of the current pulses can be extracted from the channel envelopes, where the channel envelopes correspond to the amplitude of the signal in a given frequency channel.

Thus, the audio signal processing path 251 generally operates to convert received sound signals into output signals 263, which can be used for delivering stimulation to a recipient in a manner that evokes perception of the sound signals.

As noted, cochlear implants electrically stimulate the auditory nerve, bypassing damaged sensory receptors and eliciting neural activation patterns that represent acoustic sounds. Although cochlear implants restore a sense of hearing to people with severe to profound deafness, many cochlear implant recipients still struggle with complicated listening situations, such as speech perception in noise and music perception.

These difficulties arise because of limited sound information that is transmitted by a cochlear implant. Generally, a cochlear implant extracts an envelope in frequency bands corresponding to each implanted electrode, and those envelopes are used to modulate fixed-rate biphasic pulse trains that are transmitted to the electrodes. The exclusive use of temporal envelopes in a limited number of frequency bands reduces temporal and spectral resolution of acoustic sound. Further, a cochlear implant can use techniques, such as frequency decomposition, which produce only a rough approximation of sound in order to provide computational efficiency. Moreover, electrical current delivered to the electrodes spreads through conductive fluid of the cochlea, thereby limiting channel independence. Consequently, the neural activation patterns evoked by cochlear implants are only a coarse approximation of those evoked by acoustic hearing.

According to example embodiments, stimulation control is provided by an implantable device. The stimulation control can be performed at an external device, and can use machine learning (e.g., artificial intelligence (AI)). The example embodiments provide a cochlear implant stimulation strategy that utilizes computational models of a healthy auditory system and an implanted auditory system to more closely emulate natural acoustic hearing.

Advances in electrode design and stimulation techniques (e.g., perimodiolar electrode arrays and focused multi-polar stimulation) enable finer temporal and spectral resolution in neural activation patterns generated by cochlear implant stimulation. Further, sophisticated models of the auditory system can predict spiking responses of auditory nerve fibers by modelling a middle ear, a travelling wave along a basilar membrane, inner hair cell transduction, auditory nerve synapses, and auditory nerve spiking behavior. Moreover, computational models of electrical hearing can predict response of the auditory nerve to electrical stimulation by modelling electrode characteristics, electrode placement, electrical current spread within a cochlea, neural activation, and temporal characteristics of auditory neurons (e.g., refractoriness, adaptation, facilitation, and accommodation). Computational models of electrical hearing can also be personalized to the individual cochlear implant recipient, by accounting for unique patterns of neural health along the cochlea, ossification or fibrosis within the cochlea, and/or patient-specific aetiological factors.

Example embodiments minimize a difference between normal hearing and electrical hearing computational models to transmit sound information that is more consistent with normal hearing and improve outcomes for cochlear implant recipients. This type of strategy has previously been infeasible due to massive computational demands of the auditory models, which prohibit real-time applications and increase power consumption. The inclusion of an auditory model adds many components to a stimulation pattern that are not present in spectrogram-based cochlear implant stimulation strategies, including onset enhancement, fundamental frequency modulation, and travelling wave dynamics. Onset enhancement and fundamental frequency modulation have been shown to improve speech perception in cochlear implant recipients when independently applied to a pulse train. Example embodiments encode onset enhancement and fundamental frequency modulation, and their interaction is consistent with the human auditory system.

In addition, example embodiments pre-compensate for temporal characteristics of neurons (e.g., refractoriness and adaptation) so that portions of a sound stimulus that are not encoded by neurons in a normal auditory system are not encoded by a cochlear implant processor. This conserves power and reduces unnecessary channel interaction by removing redundant pulses.

In an example embodiment, a Deep Neural Network (DNN) or other machine learning model can be trained to generate stimulation patterns that minimize a difference between an acoustic hearing neurogram and an electrically-evoked neural excitation pattern. In an embodiment, computational models can include models for auditory periphery (e.g., the spiral ganglion neuron activity in the auditory nerve). However, in other embodiments, the computational models can model more central processes, such as the auditory brainstem, inferior colliculus, or auditory midbrain.

In some embodiments, the processing capabilities of neural networks are leveraged to deliver higher resolution of auditory information adjusted for personal characteristics of a recipient. In an example embodiment, a neural network is deployed to deliver electrical hearing stimulation, having been trained according to both the recipient's characteristic way of using electric hearing and a standard reference model of “normal” hearing. Further, example embodiments of the present invention can accommodate a wide range of electrical stimulation modes, including focused multi-polar and other sensory electrical stimulation therapies which require accounting for a recipient-specific electric model against a normal response model to non-electrical stimulation.

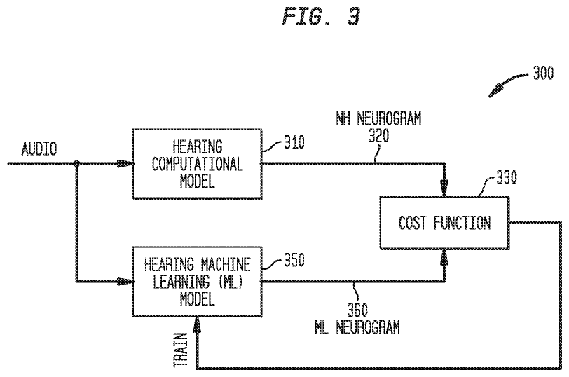

With reference to FIG. 3, shown is a functional block diagram illustrating a method 300 of training a hearing machine learning (ML) model for use with certain techniques presented herein. Initially, a training set of audio signals or audio samples is provided to a hearing computational model 310. The hearing computational model can be a computational model of a normal-hearing cochlea. These types of models are often computationally expensive and not practical to implement on a hearing aid or cochlear implant sound processor.

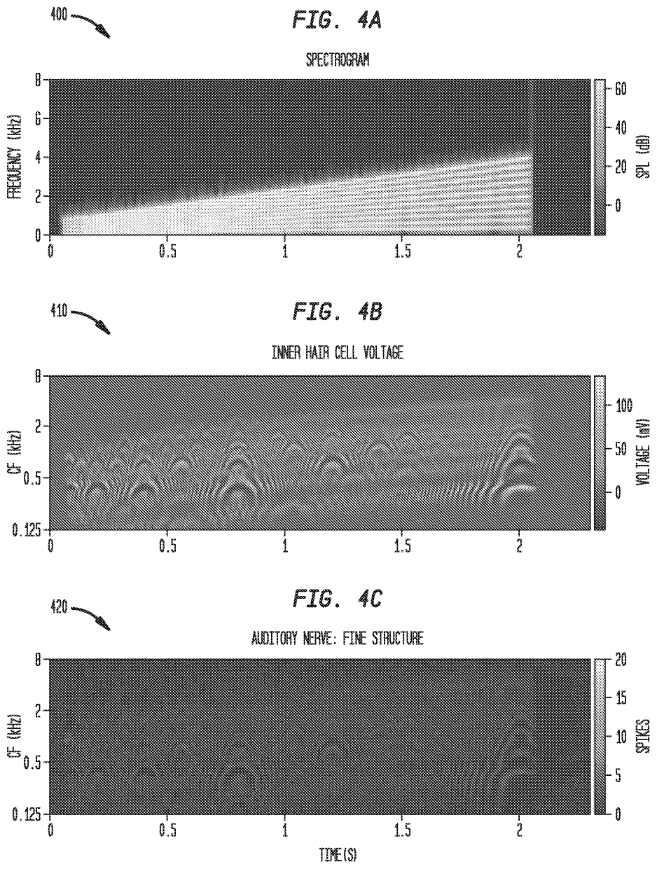

Hearing computational model 310 processes the audio signals and produces an output indicating a firing or activation pattern of neurons in an auditory nerve for normal hearing represented as a neurogram (e.g., normal hearing (NH) neurogram 320 as viewed in FIG. 3). With reference to FIGS. 4A-4C , FIG. 4A illustrates a spectrogram 400 for a rising complex tone. The spectrogram plots time across an X-axis, frequency of the tone across a first Y-axis, and power level (dB) of the tone across a second opposing Y-axis, where the power level is indicated by the shading. FIG. 4B illustrates a neurogram 410 of inner hair cell voltage for the rising complex tone. Neurogram 410 plots time across an X-axis, characteristic frequency of the tone across a first Y-axis, and voltage (in millivolts) of the inner hair across a second opposing Y-axis, where the voltage is represented by the shading. FIG. 4C illustrates a neurogram 420 of fine structure of an auditory nerve for the rising complex tone. Neurogram 420 plots time across an X-axis, characteristic frequency of the tone across a first Y-axis, and a quantity of spikes across a second opposing Y-axis, where the number of spikes is represented by the shading. Hearing computational model 310 can generate NH neurogram 320 in the form of neurogram 410 and/or neurogram 420 for use with example embodiments as described below. The hearing computational model can employ any conventional or other models of the auditory system that can predict the spiking responses of auditory nerve fibres by modelling the middle ear, the travelling wave along the basilar membrane, inner hair cell transduction, auditory nerve synapses, and auditory nerve spiking behaviour. Spectrogram 400 is typically used in conventional cochlear implant processors, while neurograms 410, 420 can be used with example embodiments and provide additional details beyond spectrogram 400.

A hearing machine learning (ML) model 350 can be trained to perform an equivalent function as hearing computational model 310 and generate a neurogram from audio signals (e.g., ML neurogram 360 as viewed in FIG. 3). Hearing ML model 350 can include any conventional or other machine learning models (e.g., mathematical/statistical models; classifiers; decision tree; random forest; feed-forward, recurrent, convolutional, convolutional recurrent, deep learning, gated, long short-term memory (LSTM), self-attention, encoder/decoder, or other neural networks; etc.) to generate the neurogram. By way of example, hearing ML model 350 can include a neural network substantially similar to the neural network described below (e.g., FIG. 6).

In a training phase, the training set of audio signals or audio samples is provided to both the hearing computational model 310 and the hearing ML model 350. The hearing ML model processes the audio signals and produces an output indicating a firing or activation pattern of neurons in an auditory nerve represented as a neurogram (e.g., ML neurogram 360 as viewed in FIG. 3). NH neurogram 320 is compared to ML neurogram 360 by a cost function 330 that provides a difference between these neurograms to train hearing ML model 350. The cost function can employ any conventional or other cost or error function (e.g., mean absolute error, L1 norm (e.g., sum of absolute differences of vector components), L2 norm (or Euclidean distance), weighting applied to differences or errors, etc.). For example, the cost function can employ a mean absolute error between data values of the NH neurogram 320 and ML neurogram 360 (e.g., a sum of absolute values of errors (or differences) divided by a sample size, etc.). The data values from neurograms 320, 360 can correspond to a same dimension (e.g., same neurons, same sampling frequency, etc.) for applying the cost function. Weights of hearing ML model 350 are adjusted via any conventional or other training technique (e.g., backpropagation, etc.) to minimize the cost function which quantifies the difference (or error) between ML neurogram 360 and NH neurogram 320. Once the difference between ML neurogram 360 and NH neurogram 320 converges (e.g., the difference remains constant or within a threshold range for a certain time period or number of training iterations), training of the hearing ML model 350 is complete. Hearing ML model 350 can be used to generate a reference neurogram representing normal hearing by example embodiments as described below.

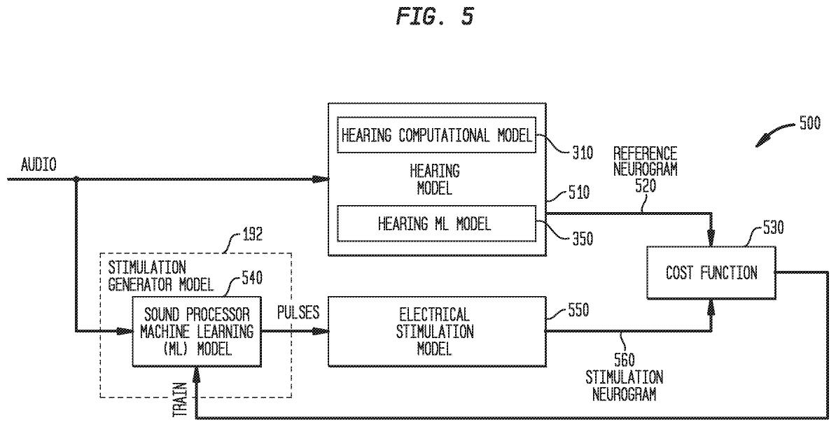

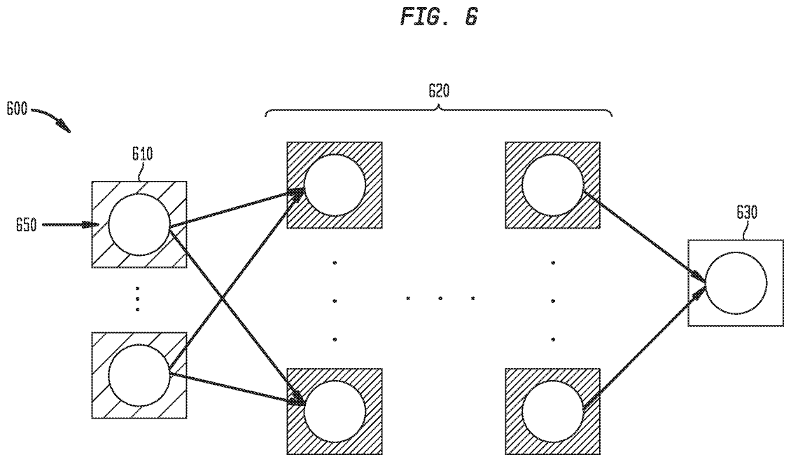

With reference to FIG. 5, shown is a functional block diagram illustrating a method 500 of training a sound processor machine learning (ML) model 540 for controlling stimulation according to certain embodiments. A training set of audio signals or audio samples are provided to a hearing model 510. The audio samples can include speech, music, broadband stimuli, and/or environmental or any other sounds. The hearing model 510 can include hearing computational model 310, or previously-trained hearing machine learning (ML) model 350 to generate a reference neurogram 520 representing normal hearing in substantially the same manner described above. The audio signals can include a microphone signal, output of a beamformer that combines multiple microphone signals, and/or audio signals from a phone or other audio accessory. Further, various pre-processing (e.g., Automatic Gain Control (AGC), noise reduction, etc.) can be applied. Hearing model 510 processes the audio signals and produces an output indicating a firing or activation pattern of neurons in an auditory nerve represented as a neurogram (e.g., reference neurogram 520 as viewed in FIG. 5). Hearing model 510 can generate reference neurogram 520 (e.g., in the form of neurogram 410 and/or neurogram 420. The audio signals can be partitioned into frames of any desired duration or length, and a neurogram or neural activation pattern can be generated for each frame.

A sound processor machine learning (ML) model 540 can be trained to produce stimulation information (e.g., pulse information, analog information, etc.) that provide a stimulation neurogram 560 similar to reference neurogram 520 representing normal hearing. Sound processor ML model 540 can employ any conventional or other machine learning models (e.g., mathematical/statistical models; classifiers; decision tree; random forest; feed-forward, recurrent, convolutional, convolutional recurrent, deep learning, gated, long short-term memory (LSTM), self-attention, encoder/decoder, or other neural networks; etc.) to generate the neurogram. By way of example, sound processor ML model 540 can employ a neural network as described below (e.g., FIG. 6).

In a training phase, the training set of audio signals or audio samples is also provided to sound processor ML model 540. The sound processor ML model 540 processes the audio signals and produces information (e.g., pulse information, analog information, etc.) that is provided to an electrical stimulation model 550. The information can include controls or characteristics of stimulation signals (e.g., pulses) that can be used by stimulator unit 142 to generate stimulation signals for implant 112. For example, the information can indicate electrical current levels and/or other characteristics for electrodes 144 of implant 112 (e.g., which electrodes are active, the electrical current level for the electrodes, a time when active, etc.) at corresponding times.

Electrical stimulation model 550 can be any conventional or other computational or machine learning model (e.g., finite element model, biophysical model, phenomenological model, neural network, etc.) of a neural response to stimulation signals delivered by a cochlear implant. Electrical stimulation model 550 models electrical field effects (e.g., electrical current spread, etc.) and a neural interface (e.g., neural thresholds for activating neurons, refractory times, etc.). This model is preferably specific to a particular cochlear implant recipient and incorporates information on the position and type of electrodes in the recipient's cochlea, patterns of neural health along the cochlea, fibrosis and ossification within the cochlea, and details about the shape and size of the cochlea and the location of a target neural population. These patient-specific details can be determined by imaging (e.g., clinical CT scan, etc.), electrophysiological measurements (e.g., electrically-evoked compound action potentials, EEG, Electrocochleography, etc.), psychophysical measurements (e.g., detection thresholds, amplitude modulation detection thresholds, masked tuning curves, etc.), or a combination thereof. For example, the position of an electrode could be estimated from radiographic imaging (CT, x-ray, etc.) or from other surgical applications that use implant telemetry (impedances) to track and estimate electrode position during implantation.

By way of example, electrical stimulation model 550 can be implemented by a neural network and trained (e.g., as described below for FIG. 6) with a training set of information and corresponding known neural responses to produce a neural response (e.g., firing or activation pattern of neurons in an auditory nerve, etc.) for an input neurogram. Electrical stimulation model 550 produces an output indicating a firing or activation pattern of neurons in an auditory nerve based on the information produced by sound processor machine learning (ML) model 540. The firing or activation pattern is represented as a neurogram (e.g., stimulation neurogram 560 as viewed in FIG. 5). Reference neurogram 520 is compared to stimulation neurogram 560 by a cost function 530 that provides a difference between these neurograms to sound processor ML model 540. The cost function can employ any conventional or other cost or error function (e.g., mean absolute error, L1 norm (e.g., sum of absolute differences of vector components), L2 norm (or Euclidean distance), weighting applied to differences or errors, etc.). For example, the cost function can employ a mean absolute error between data values of the reference neurogram 520 and stimulation neurogram 560 (e.g., a sum of absolute values of errors (or differences) divided by a sample size, etc.). The data values from neurograms 520, 560 can correspond to a same dimension (e.g., same neurons, same sampling frequency, etc.) for applying the cost function. Weights of sound processor ML model 540 are adjusted (e.g., via backpropagation, etc.) to minimize the cost function which quantifies the difference (or error) between reference neurogram 520 and stimulation neurogram 560. Once the difference between the reference neurogram and stimulation neurogram converges (e.g., the difference remains constant or within a threshold range for a certain time period or number of training iterations), training is complete and sound processor ML model 540 can be used in (e.g., stimulation generator model 192 of) a sound processor of example embodiments to process audio signals and produce information to control stimulator unit 142 to produce and apply stimulation signals for the stimulation. Thus, in the training phase, sound processor ML model 540 is trained so that the stimulation neurogram generated from information produced by the sound processor ML model is a close approximation to the reference neurogram representing normal hearing. Stimulation ML learning model 540 can be deployed to a device by providing the weights of the trained model.

By way of example, sound processor ML model 540 can employ a neural network. An example neural network 600 is illustrated in FIG. 6. Neural network 600 can include an input layer 610, one or more intermediate layers 620 (e.g., including any hidden layers), and an output layer 630. Each layer includes one or more neurons 650, where the input layer neurons receive input (e.g., audio signals or audio samples), and can be associated with weight values. The neurons of the intermediate and output layers are connected to one or more neurons of a preceding layer, and receive as input the output of a connected neuron of the preceding layer. Each connection is associated with a weight value, and each neuron produces an output based on a weighted combination of the inputs to that neuron. The output of a neuron can further be based on a bias value for certain types of neural networks (e.g., recurrent types of neural networks, etc.).

The weight (and bias) values can be adjusted based on various training techniques. For example, the machine learning of the neural network can be performed using a training set of data as input and corresponding known or reference outputs, where the neural network attempts to produce the provided output and uses an error from the output (e.g., difference between produced and known outputs) to adjust weight (and bias) values (e.g., via backpropagation or other training techniques).

With respect to sound processor machine learning (ML) model 540, the reference output corresponds to reference neurogram 520 representing normal hearing. In this case, the weights of sound processor ML model 540 are adjusted to provide appropriate information to electrical stimulation model 550 to produce stimulation neurogram 560 matching or approximating normal hearing of reference neurogram 520. The difference between the reference and stimulation neurograms are provided for adjusting weights of the sound processor ML model as described above.

In some embodiments, feature vectors can be extracted from the training set input data and used for the training as input as described below, while their known or reference corresponding outputs can be used for the training as known or reference output. A feature vector can include any suitable features of the training set input data. For example, features of audio signals can include fundamental or other frequency, pitch, amplitude or intensity, spectrogram (magnitude and/or phase), mel-frequency cepstral coefficients etc.

The output layer of the neural network indicates the resulting output (e.g., pulse information, etc.) for input data. The output layer neurons can further indicate a probability for the resulting output.

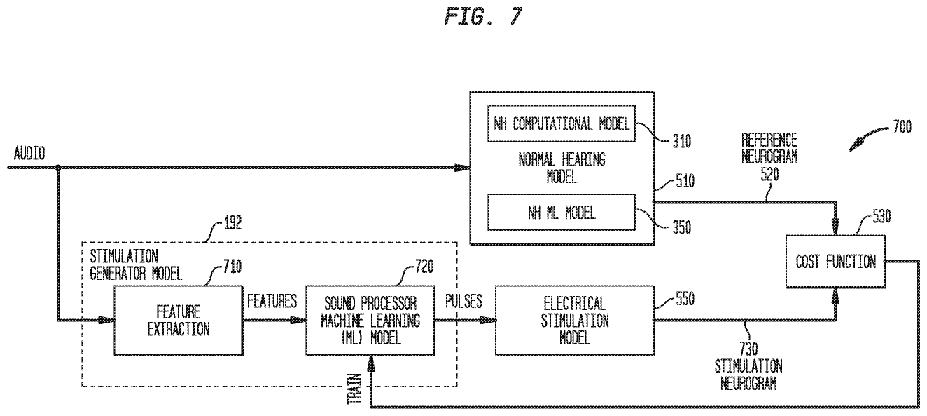

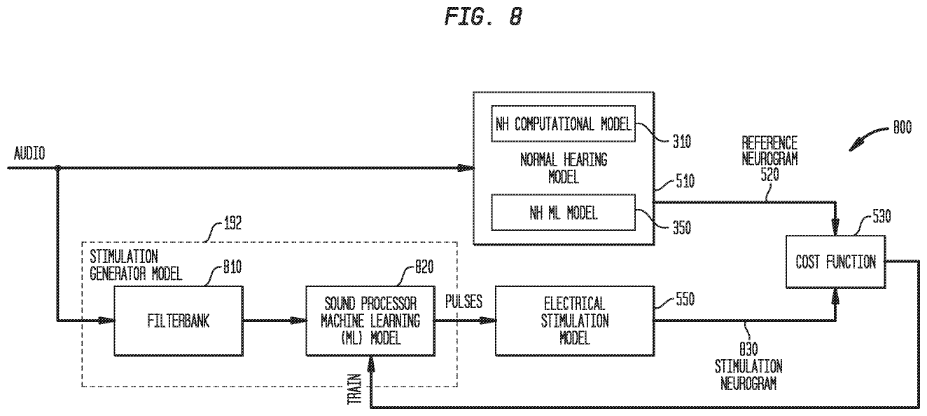

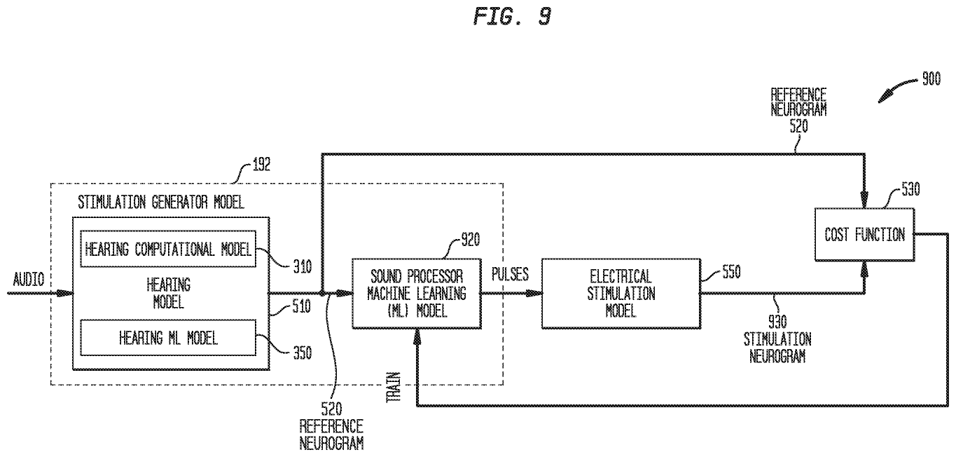

In some embodiments, a signal processing path is split into separate modules to improve efficiency. With reference to FIG. 7, shown is a functional block diagram illustrating a method 700 of training a sound processor machine learning (ML) model using feature extraction applied to an input audio signal according to certain embodiments. An example feature extraction process includes a Fast Fourier Transform (FFT) operating on a sliding time window. The FFT basically transforms audio signals from the time domain to the frequency domain. A training set of audio signals or audio samples is provided to hearing model 510. The audio samples can include speech, music, broadband stimuli, and/or environmental or any other sounds. The hearing model can include hearing computational model 310 or previously trained hearing machine learning (ML) model 350 to generate a reference neurogram 520 representing normal hearing in substantially the same manner described above. The audio signals can include a microphone signal, output of a beamformer that combines multiple microphone signals, and/or audio signals from a phone or other audio accessory. Further, various pre-processing (e.g., Automatic Gain Control (AGC), noise reduction, etc.) can be applied. Hearing model 510 processes the audio signals and produces an output indicating a firing or activation pattern of neurons in an auditory nerve represented as a neurogram (e.g., reference neurogram 520 as viewed in FIG. 7). Hearing model 510 can generate reference neurogram 520 in the form of neurogram 410 and/or neurogram 420 as described above. The audio signals can be partitioned into frames of any desired duration or length, and a neurogram or neural activation pattern can be generated for each frame.

A sound processor machine learning (ML) model 720 can be trained to produce stimulation signals that provide a neurogram similar to reference neurogram 520 representing normal hearing. Sound processor ML model 720 can employ any conventional or other machine learning models (e.g., mathematical/statistical models; classifiers; decision tree; random forest; feed-forward, recurrent, convolutional, convolutional recurrent, deep learning, gated, long short-term memory (LSTM), self-attention, encoder/decoder, or other neural networks; etc.) to generate the neurogram. By way of example, sound processor ML model 720 can employ a neural network as described above (e.g., FIG. 6).

In a training phase, the training set of audio signals or audio samples is also provided to feature extraction module 710 to extract features from the audio signals. The features can include any desired features or properties (e.g., spectrogram, features pertaining to normal hearing, etc.). In an embodiment, the feature extraction module can perform a Fast Fourier Transform (FFT) on the audio signals to extract features therefrom. The FFT basically transforms the audio signals from the time domain to the frequency domain. The extracted features can include the output of the FFT, magnitude, phase, frequency, mel-frequency cepstral coefficients (MFCCs), and/or any other signal features. The extracted features are provided to sound processor ML model 720. Sound processor ML model 720 processes the extracted features and produces information that is provided to electrical stimulation model 550. For example, the information can indicate electrical current levels and/or other characteristics for electrodes 144 of implant 112 (e.g., which electrodes are active, the electrical current level for the electrodes, a time when active, etc.) at corresponding times as described above. The electrical stimulation model can be any conventional or other computational or machine learning model (e.g., finite element model, biophysical model, phenomenological model, neural network, etc.) of a neural response to stimulation signals delivered by a cochlear implant and is substantially similar to the electrical stimulation model described above.

Electrical stimulation model 550 produces an output indicating a firing or activation pattern of neurons in an auditory nerve based on the information produced by sound processor machine learning (ML) model 720. The firing or activation pattern is represented as a neurogram (e.g., stimulation neurogram 730 as viewed in FIG. 7). Reference neurogram 520 is compared to stimulation neurogram 730 by cost function 530 that determines and provides a difference between these neurograms to sound processor machine learning (ML) model 720 in substantially the same manner described above. Weights of sound processor ML model 720 are adjusted (e.g., via backpropagation, etc.) to minimize the cost function which quantifies the difference (or error) between reference neurogram 520 and stimulation neurogram 730 in substantially the same manner described above.

Once the difference between reference neurogram 520 and stimulation neurogram 730 converges (e.g., remains constant or within a threshold range for a certain time period or number of training iterations), training is complete and feature extraction module 710 and sound processor ML model 720 can be used in (e.g., stimulation generator model 192 of) a sound processor of example embodiments to extract and process audio signal features and produce information to control stimulator unit 142 to produce and apply stimulation signals for the stimulation.

Thus, in the training phase, the reference output for sound processor machine learning (ML) model 720 corresponds to reference neurogram 520 representing normal hearing. In this case, the weights of sound processor ML model 720 are adjusted to provide appropriate information to electrical stimulation model 550 to produce stimulation neurogram 730 matching or approximating normal hearing of reference neurogram 520. The difference between the reference and stimulation neurograms are provided for adjusting weights of sound processor ML model 720 as described above. Accordingly, sound processor ML model 720 is trained so that the stimulation neurogram generated from information produced by sound processor ML model 720 is a close approximation to the reference neurogram representing normal hearing. Stimulation ML learning model 720 can be deployed to a device by providing the weights of the trained model.

In some embodiments, a filterbank process can serve as a feature extractor and be applied to the audio signal. With reference to FIG. 8, shown is a functional block diagram illustrating a method 800 of training a sound processor machine learning (ML) model with another feature extraction applied to an input audio signal according to certain embodiments. An example feature extraction process includes a filterbank process. A set of training audio signals or audio samples are provided to hearing model 510. The audio samples can include speech, music, broadband stimuli, and/or environmental or any other sounds. The hearing model can include hearing computational model 310 or previously trained hearing machine learning (ML) model 350 to generate a reference neurogram 520 representing normal hearing in substantially the same manner described above. The audio signals can include a microphone signal, output of a beamformer that combines multiple microphone signals, and/or audio signals from a phone or other audio accessory. Further, various pre-processing (e.g., Automatic Gain Control (AGC), noise reduction, etc.) can be applied. Hearing model 510 processes the audio signals and produces an output indicating a firing or activation pattern of neurons in an auditory nerve represented as a neurogram (e.g., reference neurogram 520 as viewed in FIG. 8). Hearing model 510 can generate reference neurogram 520 in the form of neurogram 410 and/or neurogram 420 as described above. The audio signals can be partitioned into frames of any desired duration or length, and a neurogram or neural activation pattern can be generated for each frame.

Sound processor machine learning (ML) model 820 can be trained to produce stimulation signals that provide a neurogram similar to reference neurogram 520 representing normal hearing. Sound processor ML model 820 can employ any conventional or other machine learning models (e.g., mathematical/statistical models; classifiers; decision tree; random forest; feed-forward, recurrent, convolutional, convolutional recurrent, deep learning, gated, long short-term memory (LSTM), self-attention, encoder/decoder, or other neural networks; etc.) to generate the neurogram. By way of example, sound processor ML model 820 can employ a neural network as described above (e.g., FIG. 6).

In a training phase, the training set of audio signals or audio samples is also provided to filterbank unit 810 to extract features from the audio signals. In an embodiment, the filterbank unit generates a suitable set of bandwidth limited channels, or frequency bins, that each includes a spectral component of the received audio. Various features can be extracted from the resulting channels or bins and provided for sound processor machine learning (ML) model 820 (e.g., the output of the filterbank processing, frequency bins or channels, frequency, phase, quantity of bins or channels, amplitudes, etc.). The filterbank unit can perform any conventional or other filterbank process (e.g., can correspond to filterbank module 256 of FIG. 2, etc.). The number of filters in the filterbank can be equal to the number of electrical stimulation channels of a cochlear implant. The extracted features are provided to sound processor ML model 820. Sound processor ML model 820 processes the extracted features and produces information that is provided to electrical stimulation model 550. For example, the information can indicate electrical current levels and/or other characteristics for electrodes 144 of implant 112 (e.g., which electrodes are active, the electrical current level for the electrodes, a time when active, etc.) at corresponding times as described above. Electrical stimulation model 550 can be any conventional or other computational or machine learning model (e.g., finite element model, biophysical model, phenomenological model, neural network, etc.) of a neural response to stimulation signals delivered by a cochlear implant and is substantially similar to the electrical stimulation model described above.

Electrical stimulation model 550 produces an output indicating a firing or activation pattern of neurons in an auditory nerve based on the information produced by sound processor machine learning (ML) model 820. The firing or activation pattern is represented as a neurogram (e.g., stimulation neurogram 830 as viewed in FIG. 8). Reference neurogram 520 is compared to stimulation neurogram 830 by cost function 530 that determines and provides a difference between these neurograms to sound processor ML model 820 in substantially the same manner described above. Weights of sound processor ML model 820 are adjusted (e.g., via backpropagation, etc.) to minimize the cost function which quantifies the difference (or error) between reference neurogram 520 and stimulation neurogram 830.

Once the difference between reference neurogram 520 and stimulation neurogram 830 converges (e.g., the difference remains constant or within a threshold range for a certain time period or number of training iterations), training is complete and filterbank module 810 and sound processor ML model 820 can be used in (e.g., stimulation generator model 192 of) a sound processor of example embodiments to extract and process audio signal features and produce information to control stimulator unit 142 to produce and apply stimulation signals for the stimulation.

Thus, in the training phase, the reference output for sound processor machine learning (ML) model 820 corresponds to reference neurogram 520 representing normal hearing. In this case, the weights of sound processor ML model 820 are adjusted to provide appropriate information to electrical stimulation model 550 to produce stimulation neurogram 830 matching or approximating normal hearing of reference neurogram 520. The difference between the reference and stimulation neurograms are provided for adjusting weights of sound processor ML model 820 as described above. Accordingly, sound processor ML model 820 is trained so that the stimulation neurogram generated from information produced by sound processor ML model 820 is a close approximation to the reference neurogram representing normal hearing. Stimulation ML learning model 820 can be deployed to a device by providing the weights of the trained model.

In an embodiment, stimulation generator model 192 can process a neurogram representing normal hearing produced from a hearing model to generate the information for the electrical stimulation model. In this case, stimulation generator model 192 is trained to remove effects of electrical stimulation on a recipient (e.g., modeled or introduced by the electrical stimulation model) to produce a neurogram matching or approximating normal hearing.

With reference to FIG. 9, shown is a functional block diagram illustrating a method 900 of training a sound processor machine learning (ML) model with a neurogram of an input audio signal according to certain embodiments. A set of training audio signals or audio samples are provided to hearing model 510. The audio samples can include speech, music, broadband stimuli, and/or environmental or any other sounds. The hearing model can include hearing computational model 310 or previously-trained hearing machine learning (ML) model 350 to generate a reference neurogram 520 representing normal hearing in substantially the same manner described above. The audio signals can include a microphone signal, output of a beamformer that combines multiple microphone signals, and/or audio signals from a phone or other audio accessory. Further, various pre-processing (e.g., Automatic Gain Control (AGC), noise reduction, etc.) can be applied. Hearing model 510 processes the audio signals and produces an output indicating a firing or activation pattern of neurons in an auditory nerve represented as a neurogram (e.g., reference neurogram 520 as viewed in FIG. 9). Hearing model 510 can generate reference neurogram 520 in the form of neurogram 410 and/or neurogram 420 as described above. The audio signals can be partitioned into frames of any desired duration or length, and a neurogram or neural activation pattern can be generated for each frame.

Sound processor machine learning (ML) model 920 can be trained to produce stimulation signals that provide a neurogram similar to reference neurogram 520 representing normal hearing. Sound processor ML model 920 can employ any conventional or other machine learning models (e.g., mathematical/statistical models; classifiers; decision tree; random forest; feed-forward, recurrent, convolutional, convolutional recurrent, deep learning, gated, long short-term memory (LSTM), self-attention, encoder/decoder, or other neural networks; etc.) to generate the neurogram. By way of example, sound processor ML model 920 can employ a neural network as described above (e.g., FIG. 6).

In a training phase, reference neurogram 520 from hearing model 510 is also provided to sound processor ML model 920. Sound processor ML model 920 processes the reference neurogram and produces information that is provided to electrical stimulation model 550. For example, the information can indicate electrical current levels and/or other characteristics for electrodes 144 of implant 112 (e.g., which electrodes are active, the electrical current level for the electrodes, a time when active, etc.) at corresponding times as described above. Electrical stimulation model 550 can be any conventional or other computational or machine learning model (e.g., finite element model, neural network, etc.) of a neural response to stimulation signals delivered by a cochlear implant and is substantially similar to the electrical stimulation model described above.

Electrical stimulation model 550 produces an output indicating a firing or activation pattern of neurons in an auditory nerve based on the information produced by sound processor machine learning (ML) model 920. The firing or activation pattern is represented as a neurogram (e.g., stimulation neurogram 930 as viewed in FIG. 9). Reference neurogram 520 is compared to stimulation neurogram 930 by cost function 530 that determines and provides a difference between these neurograms to sound processor ML model 920 in substantially the same manner described above. Weights of sound processor ML model 920 are adjusted (e.g., via backpropagation, etc.) to minimize the cost function which quantifies the difference (or error) between reference neurogram 520 and stimulation neurogram 930.

Once the difference between reference neurogram 520 and stimulation neurogram 930 converges (e.g., the difference remains constant or within a threshold range for a certain time period or number of training iterations), training is complete and hearing model 510 and sound processor ML model 920 can be used in (e.g., stimulation generator model 192 of) a sound processor of example embodiments to process audio signals and produce information in substantially the same manner described above. The information controls stimulator unit 142 to produce and apply stimulation signals for the stimulation.

Thus, in the training phase, the reference output for sound processor machine learning (ML) model 920 corresponds to reference neurogram 520 representing normal hearing. In this case, the weights of sound processor ML model 920 are adjusted to provide appropriate information to electrical stimulation model 550 to produce stimulation neurogram 930 matching or approximating normal hearing of reference neurogram 520. The difference between the reference and stimulation neurograms are provided for adjusting weights of sound processor ML model 920 as described above. Accordingly, sound processor ML model 920 is trained so that the stimulation neurogram generated from information produced by sound processor ML model 920 is a close approximation to the reference neurogram representing normal hearing. In other words, sound processor ML model 920 is trained to remove effects of electrical stimulation on a recipient (e.g., modeled or introduced by the electrical stimulation model) to produce a neurogram matching or approximating normal hearing. Stimulation ML learning model 920 can be deployed to a device by providing the weights of the trained model.

In some embodiments, noise reduction capability is provided in addition to emulating behavior of a normal hearing cochlea. In this case, during training, a hearing model is provided with a clean audio signal to produce the reference neurogram representing normal hearing, while a noisy audio signal (e.g., noise is added to the clean audio signal) is provided to generate a neurogram for training the stimulation generator model. The stimulation generator model is trained to minimize a difference between the clean reference neurogram and a resulting stimulation neurogram from a noisy signal. The stimulation generator model thus performs noise reduction and removes the effects of electrical stimulation on a recipient (modeled or introduced by the electrical stimulation generator model).

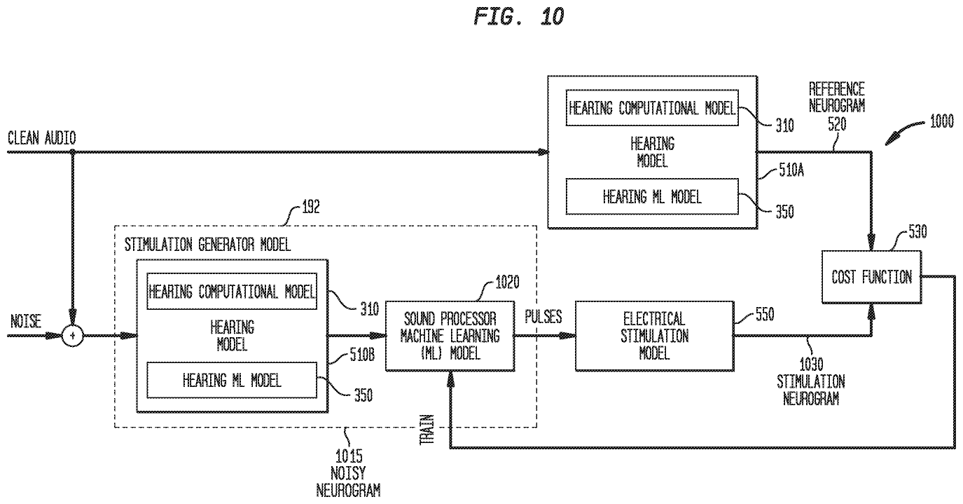

With reference to FIG. 10, shown is a functional block diagram illustrating a method 1000 of training a sound processor machine learning (ML) model to reduce noise according to certain embodiments. A set of clean training audio signals or audio samples are provided to a first hearing model 510A. The audio samples can include speech, music, broadband stimuli, and/or environmental or any other sounds. The set of clean audio signals are also provided to a mixer 1010 that introduces noise to produce noisy signals. The noise can include any type of noise (e.g., from a surrounding environment, synthetic noise, babble noise, reverberation or other convolutional noise, echoes, cafe/restaurant noise, etc.). The noisy signals are provided to a second hearing model 510B. Hearing models 510A, 510B can include hearing computational model 310 or previously-trained hearing machine learning (ML) model 350 to generate a neurogram representing normal hearing in substantially the same manner described above. The clean audio signals (prior to introduction of noise) can include a microphone signal, output of a beamformer that combines multiple microphone signals, and/or audio signals from a phone or other audio accessory. Hearing model 510A processes the clean audio signals and produces an output indicating a firing or activation pattern of neurons in an auditory nerve represented as a neurogram (e.g., reference neurogram 520 as viewed in FIG. 10). Hearing model 510A can generate reference neurogram 520 in the form of neurogram 410 and/or neurogram 420 as described above. Similarly, hearing model 510B processes the noisy audio signals and produces an output indicating a firing or activation pattern of neurons in an auditory nerve represented as a neurogram (e.g., noisy neurogram 1015 as viewed in FIG. 10). Hearing model 510B can generate noisy neurogram 1015 in the form of neurogram 410 and/or neurogram 420 as described above. The audio signals can be partitioned into frames of any desired duration or length, and a neurogram or neural activation pattern can be generated for each frame as described above.

A sound processor machine learning (ML) model 1020 can be trained to produce stimulation signals that provide a neurogram similar to reference neurogram 520 representing normal hearing. Sound processor ML model 1020 can employ any conventional or other machine learning models (e.g., mathematical/statistical models; classifiers; decision tree; random forest; feed-forward, recurrent, convolutional, convolutional recurrent, deep learning, gated, long short-term memory (LSTM), self-attention, encoder/decoder, or other neural networks; etc.) to generate the neurogram. By way of example, sound processor ML model 1020 can employ a neural network as described above (e.g., FIG. 6).

In a training phase, noisy neurogram 1015 from hearing model 510B is provided to sound processor ML model 1020. Sound processor ML model 1020 processes the noisy neurogram and produces information that is provided to electrical stimulation model 550. For example, the information can indicate electrical current levels and/or other characteristics for electrodes 144 of implant 112 (e.g., which electrodes are active, the electrical current level for the electrodes, a time when active, etc.) at corresponding times as described above. Electrical stimulation model 550 can be any conventional or other computational or machine learning model (e.g., finite element model, neural network, etc.) of a neural response to stimulation signals delivered by a cochlear implant and is substantially similar to the electrical stimulation model described above.

Electrical stimulation model 550 produces an output indicating a firing or activation pattern of neurons in an auditory nerve based on the information produced by sound processor machine learning (ML) model 1020. The firing or activation pattern is represented as a neurogram (e.g., stimulation neurogram 1030 as viewed in FIG. 10). Reference neurogram 520 (produced from clean audio signals) is compared to stimulation neurogram 1030 (produced from the noisy audio signals) by cost function 530 that determines and provides a difference between these neurograms to sound processor ML model 1020 in substantially the same manner described above. Weights of sound processor ML model 1020 are adjusted (e.g., via backpropagation, etc.) to minimize the cost function which quantifies the difference (or error) between reference neurogram 520 and stimulation neurogram 1030.

Once the difference between reference neurogram 520 and stimulation neurogram 1030 converges (e.g., the difference remains constant or within a threshold range for a certain time period or number of training iterations), training is complete and hearing model 510A and sound processor ML model 1020 can be used in (e.g., stimulation generator model 192 of) a sound processor of example embodiments to process audio signals and produce information in substantially the same manner described above. The information controls stimulator unit 142 to produce and apply stimulation signals for the stimulation.

Thus, in the training phase, the reference output for sound processor machine learning (ML) model 1020 corresponds to reference neurogram 520 representing normal hearing. In this case, the weights of sound processor ML model 1020 are adjusted to provide appropriate information to electrical stimulation model 550 to produce stimulation neurogram 1030 matching or approximating normal hearing of reference neurogram 520. The difference between the reference and stimulation neurograms are provided for adjusting weights of sound processor ML model 1020 as described above. Accordingly, sound processor ML model 1020 is trained so that the stimulation neurogram generated from information produced by sound processor ML model 1020 based on noisy audio signals is a close approximation to the reference neurogram representing normal hearing of clean audio signals. In other words, sound processor ML model 1020 is trained to remove noise and effects of electrical stimulation on a recipient (e.g., modeled or introduced by the electrical stimulation model) to produce a neurogram matching or approximating normal hearing of a clean audio signal. Stimulation ML learning model 1020 can be deployed to a device by providing the weights of the trained model.

The example embodiments can be used with any stimulation mode, although commercial cochlear implants typically use monopolar stimulation. Spatial resolution of electrical stimulation can be controlled, for example, through the use of different electrode configurations for a given stimulation channel to activate nerve cell regions of different widths. Monopolar stimulation, for instance, is an electrode configuration where for a given stimulation channel the electrical current is “sourced” via one of the intra-cochlea electrodes 144, but the electrical current is “sunk” by an electrode outside of the cochlea, sometimes referred to as the extra-cochlear electrode (ECE) 139 (FIG. 1D). Monopolar stimulation typically exhibits a large degree of electrical current spread (i.e., wide stimulation pattern) and, accordingly, has a low spatial resolution. Other types of electrode configurations, such as bipolar, tripolar, focused multi-polar (FMP), a.k.a. “phased-array” stimulation, etc. typically reduce the size of an excited neural population by “sourcing” the electrical current via one or more of the intra-cochlear electrodes 144, while also “sinking” the electrical current via one or more other proximate intra-cochlear electrodes. Bipolar, tripolar, focused multi-polar and other types of electrode configurations that both source and sink electrical current via intra-cochlear electrodes are generally and collectively referred to herein as “focused” stimulation. Focused stimulation typically exhibits a smaller degree of electrical current spread (i.e., narrow stimulation pattern) when compared to monopolar stimulation and, accordingly, has a higher spatial resolution than monopolar stimulation. Likewise, other types of electrode configurations, such as double electrode mode, virtual channels, wide channels, defocused multi-polar, etc. typically increase the size of an excited neural population by “sourcing” the electrical current via multiple neighboring intra-cochlear electrodes.

An example embodiment can utilize a more advanced stimulation model in the form of focused multi-polar stimulation. The electrical stimulation model calculates a neurogram, given multi-polar information derived from channel amplitudes (for a stimulation channel) provided by a sound processor machine learning (ML) model. Stimulation generator model 192 is trained to remove effects of electrical stimulation on a recipient (e.g., modeled or introduced by the electrical stimulation model) to produce a neurogram matching or approximating normal hearing.

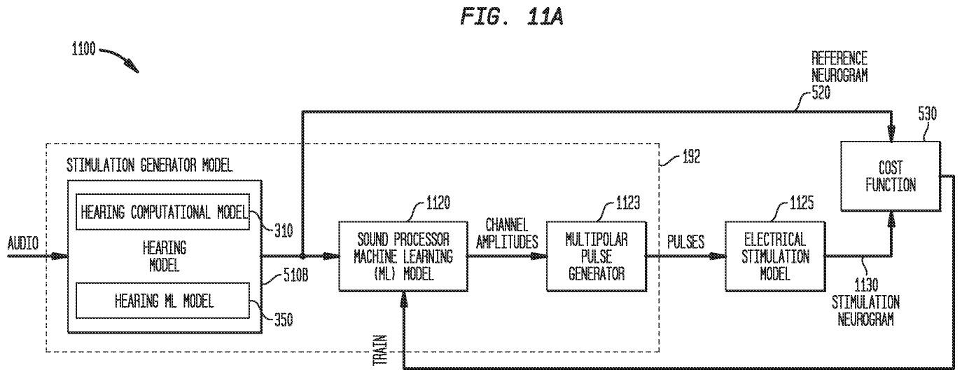

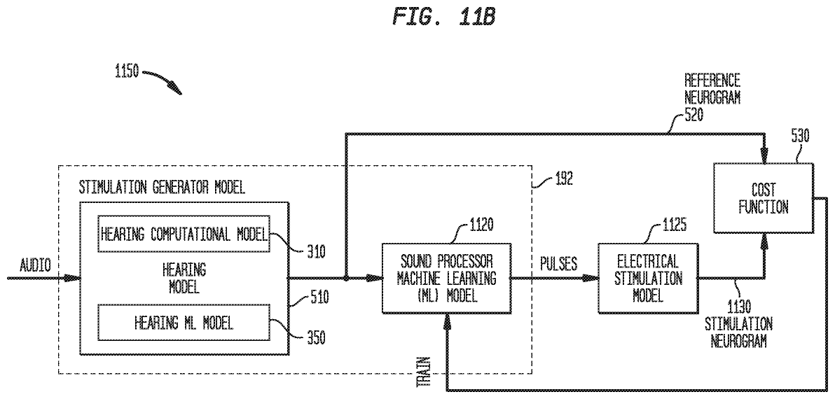

With reference to FIG. 11A, shown is a functional block diagram illustrating a method 1100 of training a sound processor machine learning (ML) model for focused multi-polar stimulation with a neurogram of an input audio signal according to certain embodiments. Focused multi-polar stimulation parameters can be individualized using electrical measurements from the cochlea (e.g. transimpedance matrices or electrically-evoked compound action potentials). A set of training audio signals or audio samples are provided to hearing model 510. The audio samples can include speech, music, broadband stimuli, and/or environmental or any other sounds. The hearing model can include hearing computational model 310 or previously-trained hearing machine learning (ML) model 350 to generate a reference neurogram 520 representing normal hearing in substantially the same manner described above. The audio signals can include a microphone signal, output of a beamformer that combines multiple microphone signals, and/or audio signals from a phone or other audio accessory. Further, various pre-processing (e.g., Automatic Gain Control (AGC), noise reduction, etc.) can be applied. Hearing model 510 processes the audio signals and produces an output indicating a firing or activation pattern of neurons in an auditory nerve represented as a neurogram (e.g., reference neurogram 520 as viewed in FIG. 11A). Hearing model 510 can generate reference neurogram 520 in the form of neurogram 410 and/or neurogram 420 as described above. The audio signals can be partitioned into frames of any desired duration or length, and a neurogram or neural activation pattern can be generated for each fame.

A sound processor machine learning (ML) model 1120 can be trained to produce channel amplitudes that are provided to focused multi-polar pulse generator 1123. The focused multi-polar pulse generator produces focused multi-polar pulses that are used to provide a neurogram similar to reference neurogram 520 representing normal hearing. Sound processor ML model 1120 can employ any conventional or other machine learning models (e.g., mathematical/statistical models; classifiers; decision tree; random forest; feed-forward, recurrent, convolutional, convolutional recurrent, deep learning, gated, long short-term memory (LSTM), self-attention, encoder/decoder, or other neural networks; etc.) to generate the neurogram. By way of example, sound processor ML model 1120 can employ a neural network as described above (e.g., FIG. 6).

In a training phase, reference neurogram 520 from hearing model 510 is also provided to sound processor ML model 1120. Sound processor ML model 1120 processes the reference neurogram and produces channel amplitudes that are provided to multi-polar pulse generator 1123. The multi-polar pulse generator generates multi-polar pulse information that is provided to electrical stimulation model 1125. For example, the multi-polar pulse information can indicate electrical current levels and/or other characteristics for specific groups of electrodes 144 of implant 112 of the stimulation channel (e.g., which electrodes are active, the electrical current level for the electrodes, a time when active, etc.) at corresponding times and provides finer control for activating smaller populations or groups of neurons. Electrical stimulation model 1125 can be any conventional or other computational or machine learning model (e.g., finite element model, neural network, etc.) of a neural response to focused multi-polar stimulation pulses delivered by a cochlear implant and is substantially similar to the electrical stimulation model described above.

Electrical stimulation model 1125 produces an output indicating a firing or activation pattern of neurons in an auditory nerve based on the multi-polar pulse information produced from channel amplitudes of sound processor machine learning (ML) model 1120. The firing or activation pattern is represented as a neurogram (e.g., stimulation neurogram 1130 as viewed in FIG. 11A). Reference neurogram 520 is compared to stimulation neurogram 1130 by cost function 530 that determines and provides a difference between these neurograms to sound processor ML model 1120 in substantially the same manner described above. Weights of sound processor ML model 1120 are adjusted (e.g., via back propagation, etc.) to minimize the cost function which quantifies the difference (or error) between reference neurogram 520 and stimulation neurogram 1130.

Once the difference between reference neurogram 520 and stimulation neurogram 1130 converges (e.g., the difference remains constant or within a threshold range for a certain time period or number of training iterations), training is complete and hearing model 510, sound processor ML model 1120, and multi-polar pulse generator 1123 can be used in (e.g., stimulation generator model 192) of a sound processor of example embodiments to process audio signals and produce channel amplitudes and multi-polar pulse information in substantially the same manner described above. The multi-polar pulse information controls stimulator unit 142 to produce and apply focused multi-polar stimulation pulses for the stimulation.

Thus, in the training phase, the reference output for sound processor machine learning (ML) model 1120 corresponds to reference neurogram 520 representing normal hearing. In this case, the weights of sound processor ML model 1120 are adjusted to provide appropriate channel amplitudes to produce multi-polar pulse information for electrical stimulation model 1125. Electrical stimulation model 1125 produces stimulation neurogram 1130 matching or approximating normal hearing of reference neurogram 520. The difference between the reference and stimulation neurograms are provided for adjusting weights of sound processor ML model 1120 as described above. Accordingly, sound processor ML model 1120 is trained so that the stimulation neurogram generated from multi-polar pulse information derived from channel amplitudes of sound processor ML model 1120 is a close approximation to the reference neurogram representing normal hearing. In other words, sound processor ML model 1120 is trained to remove effects of focused multi-polar stimulation on a recipient (e.g., modeled or introduced by the electrical stimulation model) to produce a neurogram matching or approximating normal hearing. Stimulation ML learning model 1120 can be deployed to a device by providing the weights (and other parameters) of the trained model.

With reference to FIG. 11B, shown is a functional block diagram illustrating a method 1150 of training a sound processor machine learning (ML) model for focused multi-polar stimulation with a neurogram of an input audio signal according to certain embodiments. Method 1150 is substantially similar to method 1100 described above, except that sound processor ML model 1120 incorporates multi-polar pulse generator 1123 and directly generates the multi-polar pulse information.

In a training phase, reference neurogram 520 from hearing model 510 is provided to sound processor ML model 1120 as described above. Sound processor ML model 1120 processes the reference neurogram and produces the focused multi-polar pulse information that is provided to electrical stimulation model 1125. Electrical stimulation model 1125 produces an output indicating a firing or activation pattern of neurons in an auditory nerve based on the multi-polar pulse information produced by sound processor ML model 1120. The firing or activation pattern is represented as a neurogram (e.g., stimulation neurogram 1130 as viewed in FIG. 11B).

Reference neurogram 520 is compared to stimulation neurogram 1130 by cost function 530 that determines and provides a difference between these neurograms to sound processor ML model 1120 in substantially the same manner described above. Weights of sound processor ML model 1120 are adjusted (e.g., via backpropagation, etc.) to minimize the cost function which quantifies the difference (or error) between reference neurogram 520 and stimulation neurogram 1130.

Once the difference between reference neurogram 520 and stimulation neurogram 1130 converges (e.g., the difference remains constant or within a threshold range for a certain time period or number of training iterations), training is complete and hearing model 510 and sound processor ML model 1120 can be used in (e.g., stimulation generator model 192 of) a sound processor of example embodiments to process audio signals and produce focused multi-polar pulse information in substantially the same manner described above. The multi-polar pulse information controls stimulator unit 142 to produce and apply focused multi-polar stimulation pulses for the stimulation as described above. Stimulation ML learning model 1120 can be deployed to a device by providing the weights of the trained model as described above.

The example embodiments can be used with any stimulation mode, where the various machine learning models of example embodiments (e.g., sound processor machine learning (ML) models 540, 720, 820, 920, 1020, 1120, etc.) can be trained to produce pulse information for one or more stimulation modes. For example, the machine learning models can be trained with training data (e.g., audio signals, neurograms, features, etc.) including stimulation model data indicating a type of stimulation model (e.g., monopolar, focused multi-polar, etc.). This additional data effectively creates separate spaces for the various stimulation modes. The machine learning models can be trained in substantially the same manner described above to map inputs (with stimulation model data) to pulse information of the space corresponding to the stimulation model indicated by the stimulation model data.

The various machine learning models of example embodiments (e.g., sound processor machine learning (ML) models 540, 720, 820, 920, 1020, 1120, etc.) can be pre-trained for a general or average cochlea (e.g., similar to hearing machine learning model 350 described above). In this case, these machine learning models are thereafter re-trained with recipient specific data to customize the machine learning models for specific recipients. This provides faster training of the machine learning models with less training data.

The generation of the information by the sound processor machine learning (ML) models can be performed on an external device (e.g., external component 104, etc.) and/or on another computing system (e.g., computing device 110, etc.) in communication with the external device. In the case of the information being generated on the other computing system (e.g., to conserve processing and/or battery life, etc.), the other computing system can send the information to the external device. The sound processor machine learning models can be trained with training data on the external device, other computing system, and/or a separate system, and deployed for use on the external device and/or other computing system. Further, the sound processor machine learning models can be dynamically or continuously updated or trained (and deployed) based on new information collected and obtained from the implant.

The sound processor machine learning (ML) models can be trained in various manners. The sound processor ML modes can be trained with training data (e.g., predetermined training data, data from simulations or actual hardware, etc.) on the external device, other computing system, and/or a separate system, and deployed for use on the external device and/or other computing system. Further, the sound processor ML modes can be dynamically or continuously updated or trained (and deployed) based on new information collected from the implant. The training can be performed by stimulation control logic 185.

In some embodiments, the sound processor machine learning (ML) models can be trained using various training data. For example, the training data can include a wide variety of audio files which provide various different scenarios. The scenarios can include real world examples. These various scenarios can be used as training data to train the sound processor ML modes to align with normal hearing.

The sound processor machine learning (ML) models can be trained using an entirety or any portion of the training data in substantially the same manner described above.

In some embodiments, the techniques presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. The electrical stimulation model can model physiological effects of (or physiological responses to) determined information which can be used to train a corresponding sound processor machine learning (ML) model to determine stimulation signals (e.g., pulses) that enable physiology to closely approximate or match normal physiological or sensory function (e.g., visual, smell, hearing, heart function, etc.) in substantially the same manner described above. Thus, techniques of the example embodiments can be applied to any system providing electrical stimulation to improve or compensate for improper or degrading physiological or sensory function in substantially the same manner described above.

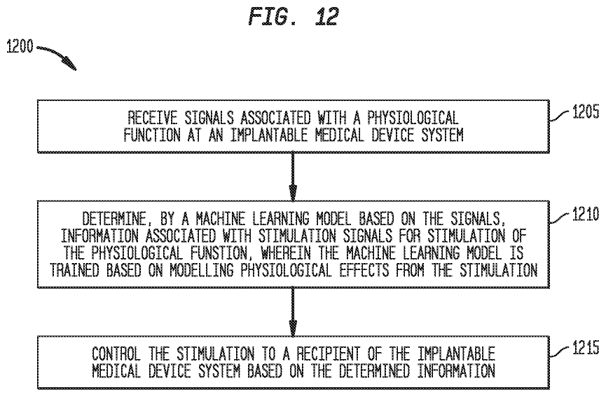

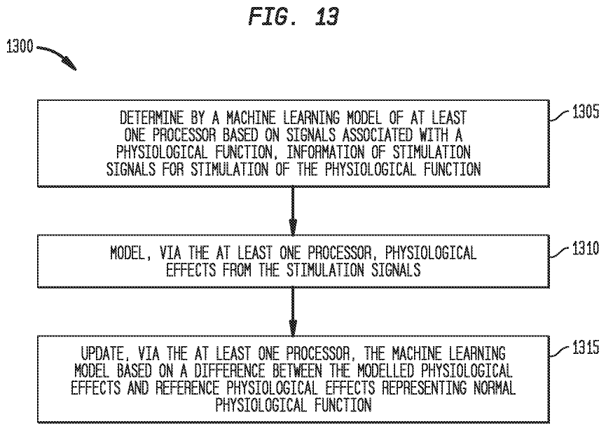

With reference now made to FIG. 12, depicted therein is a flowchart of a method 1200 for implementing the techniques of the present disclosure. Method 1200 begins in operation at 1205, which can include receiving signals associated with a physiological function at an implantable medical device system. At 1210, the method can include determining, by a machine learning model based on the signals, information for stimulation signals for stimulation of the physiological function. The machine learning model is trained based on modelling physiological effects from the stimulation. At 1215, the method can include controlling the stimulation to a recipient of the implantable medical device system based on the determined information. Accordingly, the method of flowchart 1200 provides for a process in which stimulation can be determined and controlled based on machine learning.

With reference now made to FIG. 13, depicted therein is a flowchart of a method 1300 for implementing the techniques of the present disclosure. Method 1300 begins in operation at 1305, which can include determining, by a machine learning model of at least one processor based on signals associated with a physiological function, information of stimulation signals for stimulation of the physiological function. At 1310, the method can include modelling, via the at least one processor, physiological effects from the stimulation signals. At 1315, the method can include updating, via the at least one processor, the machine learning model based on a difference between the modelled physiological effects and reference physiological effects representing normal physiological function. Accordingly, the method of flowchart 1300 provides for a process for training a machine learning model to control stimulation.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method, comprising:
receiving signals associated with a physiological function at an implantable medical device system;
determining, by a machine learning model based on the signals, information associated with stimulation signals for stimulation of the physiological function, wherein the machine learning model is trained based on modelling physiological effects from the stimulation; and
controlling the stimulation to a recipient of the implantable medical device system based on the determined information.

2. (canceled)

3. (canceled)

4. The method of claim 1, wherein the physiological function includes hearing, and wherein the machine learning model is trained to minimize a difference between a normal activation pattern for neurons in an auditory nerve and a stimulation activation pattern generated based on the determined information.

5. The method of claim 4, wherein the machine learning model is trained until a cost function indicating an error between the normal activation pattern and the stimulation activation pattern converges.

6. The method of claims 4, wherein the stimulation activation pattern is generated from the modelling of the physiological effects of the determined information.

7. The method of claim 4, wherein the physiological effects include an effect on a cochlea of stimulation signals produced based on the determined information.

8. The method of claim 4, wherein the normal and stimulation activation patterns are each represented by a neurogram indicating inner hair cell voltage.

9. The method of claim 4, wherein the normal activation pattern and the stimulation activation pattern are each represented by a neurogram indicating spikes in an auditory nerve fine structure.

10. The method of claim 4, wherein the normal activation pattern and the stimulation activation pattern are each represented by a neurogram indicating central processes of an auditory system.

11. The method of claim 1, further comprising:
extracting one or more features from the signals, wherein the machine learning model determines the information based on the extracted features.

12. (canceled)

13. (canceled)

14. The method of claim 1, wherein the machine learning model determines the information based on an activation pattern for neurons in an auditory nerve from audio signals or from audio signals combined with noise.

15. (canceled)

16. (canceled)

17. (canceled)

18. The method of claim 1, wherein the machine learning model is pre-trained for average physiological function and re-trained with recipient specific data.

19. The method of claim 1, wherein the modelling of the physiological effects is specific to the recipient.

20. One or more non-transitory computer readable storage media comprising instructions that, when executed by one or more processors, cause the one or more processors to:
receive signals associated with a physiological function at an implantable medical device system;
determine, by a machine learning model based on the signals, information of stimulation signals for stimulation of the physiological function, wherein the machine learning model is trained based on modelling physiological effects from the stimulation; and
control the stimulation to a recipient of the implantable medical device system based on the determined information.

21. (canceled)

22. (canceled)

23. The one or more non-transitory computer readable storage media of claim 20, wherein the physiological function includes hearing, and wherein the machine learning model is trained to minimize a difference between a normal activation pattern for neurons in an auditory nerve and a stimulation activation pattern generated based on the determined information.

24. (canceled)

25. The one or more non-transitory computer readable storage media of claim 23, wherein the stimulation activation pattern is generated from the modelling of the physiological effects of the determined information.

26. The one or more non-transitory computer readable storage media of claim 23, wherein the physiological effects include an effect on a cochlea of stimulation signals produced based on the determined information.

27. (canceled)

28. (canceled)

29. (canceled)

30. (canceled)

31. (canceled)

32. (canceled)

33. An implantable medical device system comprising:
memory for storing data; and
one or more processors, wherein the one or more processors are configured to:
receive signals associated with a physiological function;
determine, by a machine learning model based on the signals, information of stimulation signals for stimulation of the physiological function, wherein the machine learning model is trained based on modelling physiological effects from the stimulation; and
control the stimulation to a recipient of the implantable medical device system based on the determined information.

34. (canceled)

35. (canceled)

36. The implantable medical device system of claim 33, wherein the machine learning model is trained to minimize a difference between a normal activation pattern for neurons in an auditory nerve and a stimulation activation pattern generated based on the determined information.

37. (canceled)

38. (canceled)

39. (canceled)

40. (canceled)

41. (canceled)

42. The implantable medical device system of claim 33, wherein the machine learning model determines the information based on an activation pattern for neurons in an auditory nerve from audio signals.

43. The implantable medical device system of claim 33, wherein the machine learning model determines the information based on an activation pattern for neurons in an auditory nerve from audio signals combined with noise.

44-57. (canceled)
